(19) United States
(12) Patent Application Publication
(10) Pub. No.: US 2014/0213558 A1
(43) Pub. Date: Jul. 31, 2014

(54) TREATMENT OF INFLAMMATORY DISEASE OR DISORDER AND COMPOSITIONS THEREFOR

(71) Applicant: Philipp Crede, Cape Town (ZA)

(72) Inventor: Philipp Crede, Cape Town (ZA)

(73) Assignee: CREDE OILS (PTY) LTD, Strand (ZA)

(21) Appl. No.: 14/240,759

(22) PCT Filed: Aug. 17, 2012
(86) PCT No.: PCT/IB2012/002058

§ 371 Date: Feb. 25, 2014

Related U.S. Application Data
(60) Provisional application No. 61/527,652, filed on Aug. 26, 2011.

(51) Int. Cl. A61K 31/122; A61K 31/59; A61K 31/355; A61K 31/202; A61K 31/616

(52) U.S. Cl. CPC A61K 31/122; A61K 31/202; A61K 31/616; A61K 31/355; A61K 31/59
USPC 514/164; 514/690; 514/560; 514/458; 514/167

(57) ABSTRACT

Embodiments of the invention relate generally to the treatment of inflammatory diseases and disorders and, more particularly, to the treatment of symptoms of inflammatory diseases and disorders using thymoquinone (TQ) alone or in combination with other compounds, including eicosapentaenoic acid (EPA). In one embodiment, the invention provides a pharmaceutical composition suitable for the treatment of at least one symptom of an inflammatory disease or disorder, comprising: an effective amount of thymoquinone; and at least one physiologically-acceptable carrier, wherein an effective amount of thymoquinone is an amount capable of reducing or preventing the at least one symptom of the inflammatory disease or disorder.

### CROSS-REFERENCE TO RELATED APPLICATIONS

This application claims the benefit of co-pending U.S. Provisional Patent Application No. 61/527,652, filed 26 Aug. 2011, which is hereby incorporated herein.

### BACKGROUND OF THE INVENTION

Inflammatory diseases and disorders, as well as diseases and disorders associated with inflammation, affect a significant portion of the world population. Such diseases and disorders include, for example, osteoarthritis, rheumatoid arthritis, psoriatic arthritis, psoriasis, multiple sclerosis, Crohn's diseases, and asthma. Symptoms of such diseases and disorders include, but are not limited to, pain, stiffness, swelling, muscle immobility, and itchiness.

### BRIEF DESCRIPTION OF THE INVENTION

In one embodiment, the invention provides a method of treating at least one symptom of an inflammatory disease or disorder in an individual in need of such treatment, the method comprising: administering to the individual an effective amount of thymoquinone.

In another embodiment, the invention provides a pharmaceutical composition suitable for the treatment of at least one symptom of an inflammatory disease or disorder, comprising: an effective amount of thymoquinone; and at least one physiologically-acceptable carrier, wherein an effective amount of thymoquinone is an amount capable of reducing or preventing the at least one symptom of the inflammatory disease or disorder.

In yet another embodiment, the invention provides for the use of thymoquinone to treat one or more symptoms of an inflammatory disease or condition.

In still another embodiment, the invention provides for the use of thymoquinone in the manufacture of a medicament for the treatment of one or more symptom of an inflammatory disease or condition selected from a group consisting of: rheumatoid arthritis, psoriasis, psoriatic arthritis, Crohn's diseases, inflammatory bowel disease, multiple sclerosis, lupus erythematosus, osteoarthritis, asthma, and diseases or disorders associated with or typified by inflammation.

### BRIEF DESCRIPTION OF THE DRAWINGS

These and other features of this invention will be more readily understood from the following detailed description of the various aspects of the invention taken in conjunction with the accompanying drawings that depict various embodiments of the invention, in which:

FIG. 1 shows a schematic of the arachidonic acid cascade by which arachidonic eicosanoids are produced.

FIG. 2 shows a schematic of omega-3 and omega-6 fatty acid eicosanoids.

It is noted that the drawings of the invention are not to scale. The drawings are intended to depict only typical aspects of the invention, and therefore should not be considered as limiting the scope of the invention.

### DETAILED DESCRIPTION OF THE INVENTION

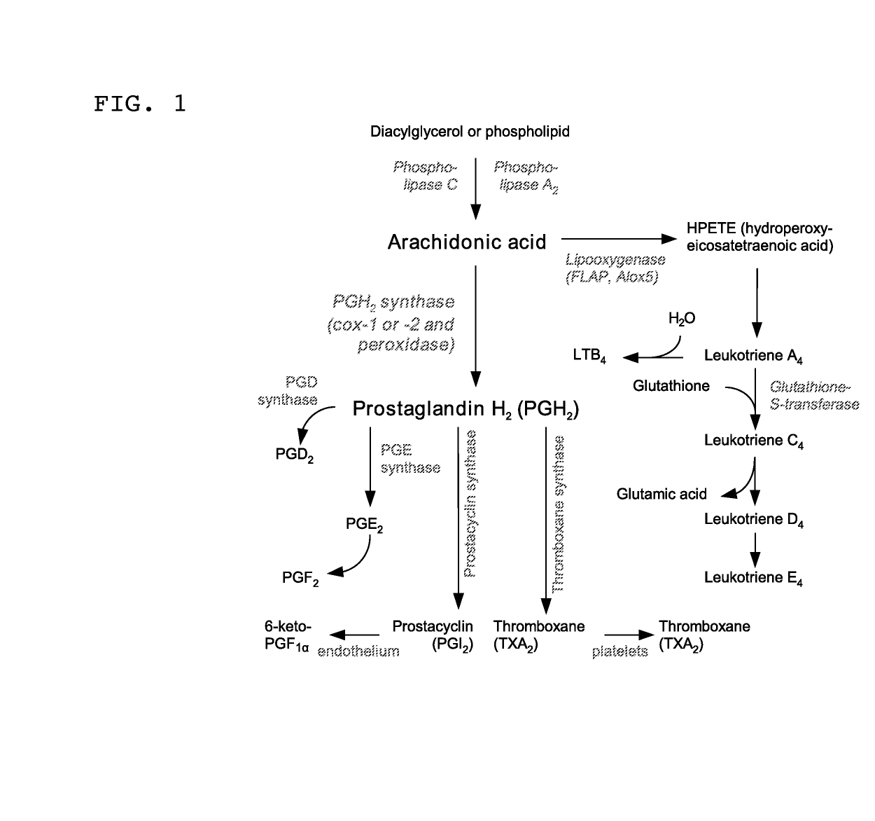

The body's inflammatory process includes an interaction of omega-3 and omega-6 essential fatty acids. For example, arachidonic acid (AA) is a 20-carbon omega-6 conditionally essential fatty acid that sits at the head of the AA cascade, shown in FIG. 1. The AA cascade comprises more than 20 signaling paths that control a wide array of bodily functions, but in particular control functions involving inflammation and the central nervous system. Most AA in the human body derives from dietary linoleic acid, an 18-carbon, 2-double-bond (18:2) omega-6 essential fatty acid found in vegetable oils and animal fats.

The body's inflammatory response involves two additional groups of dietary essential fatty acids, which form cascades that parallel and compete with the AA cascade. Eicosapentaenoic acid (EPA) is a 20:5 omega-3 essential fatty acid forming an important competing cascade. EPA is found in oily fish or derived from dietary alpha-linolenic acid, which may be found in hemp oil and flax oil. Dihomo-γ-linolenic acid (DGLA) is a 20:3 omega-6 essential fatty acid forming another competing cascade. DGLA is derived from dietary γ-linolenic acid (GLA) found, for example, in borage oil.

These competing cascades soften the inflammatory effects of AA and its products. Low dietary intake of EPA and GLA, and particularly the omega-3 EPA, is associated with a variety of inflammation-related diseases. The average human diet has, over the course of our history, tended to include less and less omega-3 fatty acids, such that the ratio of omega-3 to omega-6 fatty acids has decreased. This has been accompanied by an increase in the rates of many diseases that involve inflammatory processes.

Eicosanoids are signalling molecules derived from essential fatty acids (EFAs). Eicosanoids are a major pathway by which the EFAs act in the body. There are four classes of eicosanoid and two or three series within each class.

A cell's outer membrane contains phospholipid fat. Each phospholipid molecule contains two fatty acids. Some of these fatty acids are 20-carbon polyunsaturated essential fatty acids—AA, EPA or DGLA. In response to a variety of inflammatory signals, these EFAs are cleaved out of the phospholipid and released as free fatty acids.

Next, the EFA is oxygenated (by either of two pathways), then further modified, yielding the eicosanoids. Cyclooxygenase (COX) oxidation removes two C═C double bonds, leading to the thromboxane (TX), prostaglandin (PG), and prostacyclin (PGI) series. Lipoxygenase oxidation removes no C═C double bonds, and leads to the leukotriene (LT) series.

After oxidation, the eicosanoids are further modified, making a series. Members of a series are differentiated by an A, B, C, etc., and are numbered by the number of double bonds, which does not change within a series. For example, cyclooxygenase action upon AA, which has 4 double bonds, leads to the series-2 thromboxanes (TXA2, TXB2, etc.), each with two double bonds. Cyclooxygenase action on EPA, which has 5 double bonds, leads to the series-3 thromboxanes (TXA3, TXB3, etc.), each with three double bonds. There are exceptions to this pattern, some of which indicate stereochemistry.

FIG. 1 shows these sequences for AA (20:4 omega-6). The sequences for EPA (20:5 omega-3) and DGLA (20:3 omega-6) are analogous.

In the AA cascade, dietary linoleic acid (18:2 omega-6) is lengthened and desaturated to form AA and esterified into phospholipid fats in the cell membrane. In response to many inflammatory stimuli, phospholipase is generated and cleaves this fat, releasing AA as a free fatty acid, which can then be oxygenated and further modified to form eicosanoids, autocrine and paracrine agents that bind receptors on the cell or its neighbors. In other cases, AA can diffuse into the cell nucleus and interact with transcription factors to control DNA transcription for cytokines and other hormones.

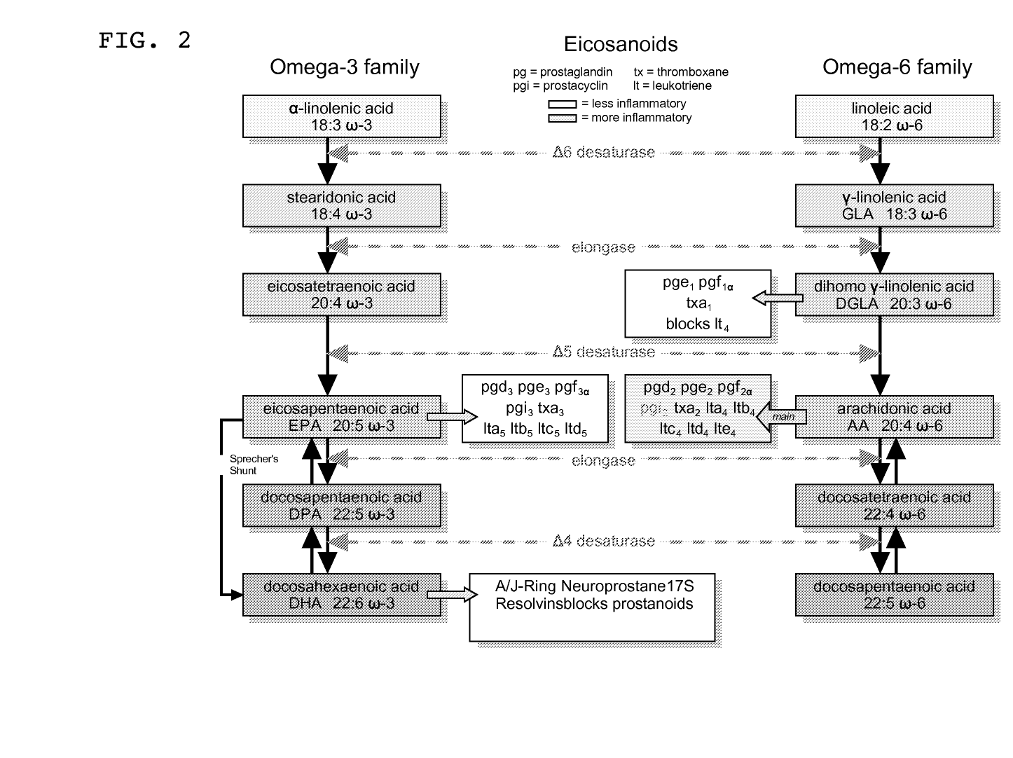

Although there are some tissue-specific differences, in general, eicosanoids from AA promote inflammation while those from GLA (via DGLA) and EPA are less inflammatory, inactive, or anti-inflammatory. Table 1 below shows the eicosanoid series derived from GLA (via DGLA), AA, and EPA. FIG. 2 shows the omega-3 and omega-6 synthesis chains, along with their eicosanoids from AA, EPA, and DGLA.

TABLE 1

| | Formula | Eicosanoid id product series | | |
|---|---|---|---|---|
| | omega | TX | | |
| | carbons:double | PG | | |
| EFA | bonds | PGI | LT | Effects |
| GLA/DGLA | omega-6 18:3 | series-1 | series-3 | less |
| | omega-6 20:3 | | | inflammatory |
| AA | omega-6 20:4 | series-2 | series-4 | more |
| | | | | inflammatory |
| EPA | omega-3 20:5 | series-3 | series-5 | less |
| | | | | inflammatory |

Dietary omega-3 and GLA counter the inflammatory effects of AA eicosanoids via displacement, competitive inhibition, and direct counteraction. Dietary omega-3 decreases tissue concentrations of AA. For example, animal studies have shown that increased dietary omega-3 results in decreased AA in brain and other tissue. Alpha-linolenic acid contributes to this by displacing linoleic acid from the elongase and desaturase enzymes that produce AA. Other studies have shown that EPA inhibits the release of AA from the cell membrane by phospholipase.

DGLA and EPA compete with AA for access to the cyclooxygenase and lipoxygenase enzymes. As a consequence, the presence of DGLA and EPA in tissues lowers the production of AA eicosanoids. For example, dietary GLA has been shown to increase tissue DGLA and lower TXB2. Likewise, EPA inhibits the production of series-2 PG and TX.

Some DGLA- and EPA-derived eicosanoids counteract their AA-derived counterparts. For example, DGLA yields PGE1, which powerfully counteracts PGE2. EPA yields the antiaggregatory prostacyclin PGI3 and also the leukotriene LTB5, which vitiates the action of the AA-derived LTB4.

In addition, resolvins are synthesized in vivo from EPA and DHA. Specifically, resolvins are produced by the COX2 pathway, especially in the presence of aspirin. Experimental evidence indicates that resolvins reduce cellular inflammation by inhibiting the production and transportation of inflammatory cells and chemicals to sites of inflammation.

Although dietary linoleic acid (18:2 omega-6) is inflammatory, it is desaturated in the body to form GLA, which is anti-inflammatory. This paradox is partially explained by the fact that linoleic acid competes with alpha-linolenic acid (18:3 omega-3) for delta-6-desaturase and thereby inhibits formation of anti-inflammatory EPA. GLA, on the other hand, does not compete for delta-6-desaturase. GLA's elongation product, DGLA, competes with 20:4 omega-3 EFAs for the delta-5-desaturase, which might suggest that GLA would be inflammatory. It is not, however, perhaps because this step is not rate-determining, as is the delta-6-desaturase step (20:4 omega-3 EFAs do not significantly accumulate in bodily lipids).

Dietary GLA leads to sharply increased DGLA in white blood cell membranes, whereas linoleic acid does not. This may reflect a lack of desaturase in white blood cells. Supplementing dietary GLA increases serum DGLA without increasing serum AA. Although some dietary GLA may eventually form AA and contribute to inflammation, animal studies indicate that this effect is small. Empirical observation of GLA effects suggest that DGLA's anti-inflammatory effects dominate.

According to embodiments of the invention, EPA and GLA may serve to reduce inflammation by, for example, inhibiting the production of more inflammatory eicosanoids and/or stimulating the production of less-inflammatory or anti-inflammatory eicosanoids. Tests on volunteers carried out according to some embodiments of the invention are described below and demonstrate an anti-inflammatory effect, particularly in embodiments involving the co-administration of TQ, EPA, and GLA. Among the omega-3 EFAs studied, EPA was shown to be more effective as an anti-inflammatory than was docosahexaenoic acid (DHA) or alpha-linolenic acid.

Thymoquinone (TQ), also referred to as 2-isopropyl-5-methylbenzo-1,4-quinone, is known to have antioxidant, analgesic, anticonvulsant, and anti-angiogenic activity. It may also produce anti-inflammatory effects, however, via one or more mechanisms. For example, TQ reduces the production of interleukin-1-beta (IL-1β), a member of the interleukin-1 cytokine family. IL-1β is an important mediator of the inflammatory response and is involved in cell proliferation, differentiation, and apoptosis. The induction of cyclooxygenase-2 (PTGS2/COX2) by IL-1β in the central nervous system contributes to inflammatory pain hypersensitivity. TQ also reduces production of tumor necrosis factor alpha, a cytokine involved in systemic inflammation.

TQ reduces the production of COX2, which converts EFAs into TXs, PGs, and PGIs, as described above. Inhibition of COX2, therefore, inhibits the production of eicosanoids that would have an inflammatory effect. TQ also reduces the production of prostaglandin E2, a powerful inflammatory eicosanoid.

Finally, TQ inhibits synthesis of 5-lipoxygenase products, such as LTs. In particular, TQ inhibits the production of LTs from AA, which, as described above, play an important role in the inflammatory response, especially in asthma.

Although it has been suggested that TQ may be useful only in treating autoimmune diseases, such as rheumatoid arthritis, it has been surprisingly discovered that TQ, alone or in combination with other actives, such as EPA, is useful in treating other, non-autoimmune conditions, including osteoarthritis.

Diseases and disorders which may be treated and/or prevented according to various embodiments of the invention include, for example, autoimmune diseases, including rheumatoid arthritis, psoriasis, psoriatic arthritis, Crohn's diseases, inflammatory bowel disease, multiple sclerosis, and lupus erythematosus, as well as diseases of inflammation, including osteoarthritis, and asthma. Other diseases or disorders associated with or typified by inflammation may also be treated and/or prevented according to various embodiments of the invention.

In one study, 18 volunteers suffering from osteoarthritis, rheumatoid arthritis, or psoriatic arthritis were given a daily dosage of 90 mg TQ, 900 mg EPA, 440 mg GLA, and 190 mg DHA contained in two 1000 mg softgel capsules for a period of eight weeks. The remainder of the 1000 mg of each capsule comprised carriers and other inactive ingredients.

All 18 participants reported a significant reduction in pain, increased mobility, and reduced morning stiffness using various measures of arthritis severity. Negative side effects were limited to upset stomach in two participants. Positive side effects, including improved hair growth, improved skin condition, and greater general wellbeing, were reported by several participants.

In a second study, 13 volunteers with psoriasis were administered the same dosages as in the first study for a period of eight weeks. Six participants reported improvement, including reduced scaling and itching. Seven participants reported no change in their condition.

The dosages in these studies are indications only. In some cases, lower dosages of TQ might be used, for example in cases where adverse long-term reactions might occur at higher doses in the 1-800 mg TQ per day range. Such lower doses may be, for example, between about 30 mg per day and about 120 mg per day. In some embodiments, the daily dose may be about 70 mg.

The dosage of EPA might be increased or decreased depending on consumer response. Increasing EPA dosage up to 3,000 mg per day may increase efficacy; decreasing the EPA dosage would increase the product's ease of use if it reduced the number of softgel capsules required on a daily basis. The daily dosage of EPA, according to some embodiments of the invention, may be between about 200 mg and about 3,000 mg.

Other TQ-containing pharmaceutical compositions may be formulated, for example, including those containing only TQ as an active ingredient and those containing TQ in combination with EPA. Other actives may also be included, including aspirin, vitamins E, D, and/or B, as well as DHA or other polyunsaturated fatty acids.

In some embodiments of the invention, high concentrations of EPA and GLA are more effective, because lower concentrations may accompany significant amounts of linoleic acid, a precursor of AA. It has also been found that the administration of pure TQ is more effective than the administration of Nigella sativa oil, from which TQ is derived, because the oil may also contain significant amounts of linoleic acid. Nevertheless, some embodiments of the invention may include N. sativa oil and/or its essential oil, either as the sole or primary active or in combination with EPA and/or GLA. The essential oil of N. sativa contains a significantly greater percentage (about 30%) of TQ than does N. sativa oil (about 0.6% TQ). Thus, N. sativa essential oil may provide a therapeutic or prophylactic effect in some embodiments of the invention, particularly in those embodiments in which one or more other actives (e.g., EPA, GLA, etc.) are also administered. Similarly, embodiments of the invention may include derivatives and/or precursors of TQ, including polymers of TQ.

Pharmaceutical compositions for oral administration, according to various embodiments of the invention, may be take any number of forms, including, for example, softgels and tablets. In the case that EPA/GLA is administered in triglyceride form, softgels are preferred. In the case that EPA/GLA is administered in ethyl ester form, tablet forms are preferred.

The pharmaceutical compositions according to various embodiments of the invention include suitable dosage forms for oral, parenteral (including subcutaneous, intramuscular, intradermal and intravenous) transdermal, bronchial or nasal administration. Thus, if a solid carrier is used, the preparation may be tableted, placed in a hard gelatin capsule in powder or pellet form, or in the form of a troche or lozenge. The solid carrier may contain conventional excipients such as binding agents, fillers, tableting lubricants, disintegrants, wetting agents and the like. The tablet may, if desired, be film coated by conventional techniques. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. Particularly useful is the administration of TQ in oral dosage formulations. The pharmaceutical compositions are prepared by conventional techniques appropriate to the desired preparation containing appropriate amounts of the active ingredient, that is, for example, TQ according to the invention. See, for example, Remington's Pharmaceutical Sciences, Mack Publishing Company, Easton, Pa., 17th edition, 1985.

In making pharmaceutical compositions containing compounds of the present invention, the active ingredient(s) will usually be mixed with a carrier, or diluted by a carrier, or enclosed within a carrier which may be in the form of a capsule, sachet, paper or other container. When the carrier serves as a diluent, it may be a solid, semi-solid or liquid material which acts as a vehicle, excipient or medium for the active ingredient. Thus, the composition can be in the form of tablets, pills, powders, lozenges, sachets, cachets, elixirs, suspensions, emulsions, solutions, syrups, aerosols (as a solid or in a liquid medium), ointments containing the active compound, soft and hard gelatin capsules, suppositories, sterile injectable solutions and sterile packaged powders.

Some examples of suitable carriers and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, methyl cellulose, methyl- and propylhydroxybenzoates, talc, magnesium stearate and mineral oil. The formulations can additionally include lubricating agents, wetting agents, emulsifying and suspending agents, preserving agents, sweetening agents or flavoring agents. The compositions of the invention may be formulated so as to provide quick, sustained or delayed release of the active ingredient after administration to the patient.

The compositions are preferably formulated in a unit dosage form, each dosage containing from about 1 mg to about 800 mg of the active ingredient. The term “unit dosage form” refers to physically discrete units suitable as unitary dosages for human subjects and other mammals, each unit containing a predetermined quantity of active material calculated to produce the desired therapeutic effect, in association with the required pharmaceutical carrier.

This written description uses examples to disclose the invention, including the best mode, and also to enable any person skilled in the art to practice the invention, including making and using any devices or systems and performing any related or incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of treating at least one symptom of an inflammatory disease or disorder in an individual in need of such treatment, the method comprising:
administering to the individual an effective amount of thymoquinone.

2. The method of claim 1, wherein the effective amount of thymoquinone is between about 1 mg/day and about 800 mg/day.

3. The method of claim 2, wherein the effective amount of thymoquinone is between about 30 mg/day and about 120 mg/day.

4. The method of claim 3, wherein the effective amount of thymoquinone is about 70 mg/day.

5. The method of claim 1, further comprising:
administering to the individual a quantity of eicosapentaenoic acid.

6. The method of claim 5, wherein the quantity of eicosapentaenoic acid is between about 200 mg/day and about 3000 mg/day.

7. The method of claim 1, wherein administering includes enterally administering the effective amount of thymoquinone to the individual.

8. The method of claim 7, wherein the effective amount of thymoquinone is contained in an oral dosage formulation.

9. The method of claim 8, wherein the oral dosage formulation further comprises at least one of the following: a quantity of EPA, a quantity of GLA, a quantity of DHA, a quantity of aspirin, a quantity of vitamin E, a quantity of vitamin D, or a quantity of vitamin B.

10. The method of claim 1, wherein the oral dosage formulation is substantially free of linoleic acid.

11. The method of claim 1, wherein the inflammatory disease or disorder is selected from a group consisting of: rheumatoid arthritis, psoriasis, psoriatic arthritis, Crohn's diseases, inflammatory bowel disease, multiple sclerosis, lupus erythematosus, osteoarthritis, asthma, and diseases or disorders associated with or typified by inflammation.

12. A pharmaceutical composition suitable for the treatment of at least one symptom of an inflammatory disease or disorder, comprising:
an effective amount of thymoquinone; and
at least one physiologically-acceptable carrier,
wherein an effective amount of thymoquinone is an amount capable of reducing or preventing the at least one symptom of the inflammatory disease or disorder.

13. The pharmaceutical composition of claim 12, wherein the effective amount of thymoquinone constitutes a dosage between about 1 mg/day and about 800 mg/day.

14. The pharmaceutical composition of claim 13, wherein the effective amount of thymoquinone constitutes a dosage between about 30 mg/day and about 120 mg/day.

15. The pharmaceutical composition of claim 14, wherein the effective amount of thymoquinone constitutes a dosage of about 70 mg/day.

16. The pharmaceutical composition of claim 12, further comprising at least one additional ingredient selected from a group consisting of: eicosapentaenoic acid, EPA, GLA, DHA, aspirin, vitamin E, vitamin D, and vitamin B.

17. The pharmaceutical composition of claim 16, wherein the at least one additional ingredient includes eicosapentaenoic acid.

18. The pharmaceutical composition of claim 17, wherein the eicosapentaenoic acid is present at a dosage between about 200 mg/day and about 3000 mg/day.

19. Use of thymoquinone to treat one or more symptom of an inflammatory disease or condition.

20. The use of claim 19, wherein the inflammatory disease or condition is selected from a group consisting of: rheumatoid arthritis, psoriasis, psoriatic arthritis, Crohn's diseases, inflammatory bowel disease, multiple sclerosis, lupus erythematosus, osteoarthritis, asthma, and diseases or disorders associated with or typified by inflammation.

21. Use of thymoquinone in the manufacture of a medicament for the treatment of one or more symptom of an inflammatory disease or condition selected from a group consisting of: rheumatoid arthritis, psoriasis, psoriatic arthritis, Crohn's diseases, inflammatory bowel disease, multiple sclerosis, lupus erythematosus, osteoarthritis, asthma, and diseases or disorders associated with or typified by inflammation.
